(19) United States
(12) Patent Application Publication
(10) Pub. No.: US 2025/0288505 A1
(43) Pub. Date: Sep. 18, 2025

(54) CONTAINER FOR REPLACEABLE FLUID COSMETIC DISPENSER

(71) Applicant: Tilt Beauty, LLC, Los Angeles, CA (US)

(72) Inventors: Aerin D. Glazer, Beverly Hills, CA (US); Samuel Dominic O'Donahue, New York, NY (US)

(21) Appl. No.: 19/082,810

(22) Filed: Mar. 18, 2025

Related U.S. Application Data
(60) Provisional application No. 63/566,756, filed on Mar. 18, 2024.

(51) Int. Cl. A61K 8/06; A61Q 1/02

(52) U.S. Cl. CPC A61K 8/06; A61Q 1/02; A61K 2800/87

(57) ABSTRACT

A cosmetic system disclosed herein includes a container with a large-radius, grippable exterior that houses a smaller, modular cosmetic dispenser that can be removed and replaced to facilitate use of the exterior container. In one aspect, the modular cosmetic dispenser may be a solid cosmetic dispenser for lipstick or the like, with the mechanical components for dispensing the cosmetic contained withing the disposable, modular dispenser. In another aspect, the modular cosmetic dispenser may be a fluid cosmetic dispenser for a nail polish or the like, with a reservoir, threaded top, and applicator brush or the like integrated into the modular dispenser. As a significant advantage, this permits the re-use of a high-quality, ergonomically designed container with disposable modular cosmetics dispensers.

### RELATED APPLICATIONS

This application claims priority to U.S. Prov. App. No. 63/566,756 filed on Mar. 18, 2024, the entire contents of which are hereby incorporated by reference.

### TECHNICAL FIELD

This disclosure relates to cosmetic dispensers, and more specifically to modular cosmetic dispensers for various solid and fluid cosmetics.

### BACKGROUND

Cosmetics are typically provided in a variety of mediums and form factors. For example, cosmetics may have various formulations for dispensing, e.g., as a fluid (such as nail polish, mascara, or lip gloss) or a solid (such as a lipstick or an eye shadow). These different formulations may also be sold for use in various packages and dispensers such as twist up tubes, click pens, stick dispensers, and so forth. However the current form factors for these cosmetics are typically compact, and can impose challenges for people with conditions that limit dexterity and fine motor skills. For example, conditions such as arthritis, Parkinson's Disease, Multiple Sclerosis, Carpal Tunnel Syndrome, Muscular Dystrophy, Cerebral Palsy, peripheral neuropathy, and tendonitis can impose challenges with tasks such as gripping, pinching, and manipulating small objects.

There remains a need for improved cosmetic dispensers suitable for use by people with dexterity challenges.

### SUMMARY

A cosmetic system disclosed herein includes a container with a large-radius, grippable exterior that houses a smaller, modular cosmetic dispenser that can be removed and replaced to facilitate use of the exterior container. In one aspect, the modular cosmetic dispenser may be a solid cosmetic dispenser for lipstick or the like, with the mechanical components for dispensing the cosmetic contained withing the disposable, modular dispenser. In another aspect, the modular cosmetic dispenser may be a fluid cosmetic dispenser for a nail polish or the like, with a reservoir, threaded top, and applicator brush or the like integrated into the modular dispenser. As a significant advantage, this permits the re-use of a high-quality, ergonomically designed container with disposable modular cosmetics dispensers.

In an aspect, a device disclosed herein includes a dispenser and an applicator that assemble into a dispensing module, where the dispenser includes: a cosmetic including a pigment in a fluid carrier, a tube with a reservoir containing the cosmetic, a first end of the dispenser including an opening to the reservoir and a threaded end portion, and a second end of the dispenser having a stem extending along an axis of the dispenser, the stem having a cross-section with a non-circular shape. The applicator may include: a top for the dispenser, the top including a threaded top portion complementary to the threaded end portion of the dispenser, where the top, when coupled to the dispenser by threading the threaded top portion to the threaded end portion, encloses the cosmetic within the reservoir of the dispenser, a wand extending from the top into the reservoir when the top is placed for use on the dispenser, and an applicator tip on the wand.

The cross-section of the stem may have a repeating radial pattern with a capture angle of less than twenty degrees. The cosmetic may include a nail polish. The cosmetic may include a mascara. The cosmetic may include an eye liner. The cosmetic may include one or more of an emulsifier, a preservative, a thickener, and an emollient. A bottom surface of the stem may be color coded with an indicia of a color of the cosmetic in the dispensing module.

In an aspect, a dispensing module disclosed herein includes a dispenser including: a reservoir, a tube on a first end of the dispenser, the tube including an opening to the reservoir and a threaded end portion, and a stem extending along an axis of the dispenser from a second end of the dispenser, the stem defining a cross-section with a repeating radial pattern having an intrinsic capture angle not greater than forty-five degrees. The dispensing module may also include an applicator including: a top for the dispenser, the top including a threaded top portion complementary to the threaded end portion of the tube, where the top, when coupled to the dispenser by threading the threaded top portion to the threaded end portion, encloses and fluidically seals the reservoir of the dispenser, a wand extending from the top into the reservoir when the top is placed for use on the dispenser, and an applicator tip on the wand.

An exterior surface of the top may define a second cross-section with a second repeating radial pattern having a capture angle less than twenty degrees to a cap for the top. The intrinsic capture angle of the repeating radial pattern of the cross-section of the stem may not be greater than twenty degrees. The intrinsic capture angle of the repeating radial pattern of the cross-section of the stem may not be greater than fifteen degrees. The dispensing module may include a fluid cosmetic in the reservoir, the fluid cosmetic including a pigment in a fluid carrier. The fluid cosmetic may include a nail polish, a mascara, or an eye liner. The fluid cosmetic may include one or more of an emulsifier, a preservative, a thickener, and an emollient. The applicator may include at least one of a brush and a swab

In an aspect, a system for dispensing fluid cosmetics disclosed herein includes a dispenser, where the dispenser includes: a cosmetic, a tube on a first end of the dispenser, the tube including a reservoir for the cosmetic, an opening to the reservoir, and a threaded end portion, and a stem on a second end of the dispenser, the stem extending along an axis of the dispenser, and the stem having a radially repeating cross-sectional shape. The system may also include an applicator including: a top for the dispenser, a threaded top portion of the top with threads complementary to the threaded end portion of the tube, where the top, when coupled to the dispenser by threading the threaded top portion to the threaded end portion, encloses the cosmetic within the reservoir of the dispenser, and a wand with an applicator tip extending from the top into the reservoir when the top is placed for use on the dispenser.

The system may include a container for the dispenser, where: the container includes a receiver shaped and sized to removably and replaceable receive the stem of the dispenser, the receiver engages the stem against linear displacement with a draw force of at least one newton along the axis of the dispenser, and the receiver has an interior cross section shaped to receive the stem with a capture angle not exceeding twenty degrees, and to rotationally fix the container to the stem when coupled thereto. The system may include a gripping region on an exterior surface of the container, the gripping region including an overmolded shell of a low durometer material with a thickness of at least one millimeter. The gripping region may have a radius along at least a portion of the gripping region not less than twenty five millimeters. The cap may include a rigid interior component shaped and sized to engage in a rotationally fixed manner with the top of the applicator, and a gripping region of relatively soft high grip material overmolded onto the rigid interior component to provide a gripping surface for a user.

In an aspect, a cosmetic dispenser disclosed herein includes: a dispensing module; a cosmetic in the dispensing module, the cosmetic including a pigment in a solid carrier; a tube on a first end of the dispensing module, the tube surrounding at least a portion of the cosmetic; an opening on the first end of the dispensing module to dispense the cosmetic; a stem extending along an axis of the dispensing module from a second end of the dispensing module, the stem having a cross-section with a non-circular shape; and a dispensing mechanism, the dispensing mechanism including a mechanical actuator configured to advance the cosmetic from the tube through the opening by creating a linear motion along the axis of the dispensing module in response to a rotational movement of the stem relative to the tube.

The cross-section of the stem may have a repeating radial pattern with a capture angle not greater than forty-five degrees. The cross-section of the stem may have a repeating radial pattern with a capture angle not greater than twenty degrees. The cross-section of the stem may have a capture angle of fifteen degrees. The solid carrier may include a wax. The solid carrier may include one or more of an oil and a glycerin. The cosmetic may be a lipstick. The cosmetic may be an eye liner or an eye shadow. A bottom surface of the stem may be color coded with an indicia of a color of the cosmetic in the dispensing module.

In an aspect, a dispensing module for dispensing solid cosmetics disclosed herein includes: a cosmetic including a pigment in a solid carrier; a tube on a first end of the dispensing module, the tube surrounding at least a portion of the cosmetic; an opening in the first end of the dispensing module; a stem extending along an axis of the dispensing module from a second end of the dispensing module, the stem defining a cross-section with a repeating radial pattern that resists rotation relative to a receiver with a complementary geometric shape; and an actuator to linearly dispense the cosmetic through the opening in response to a rotation of the stem relative to the tube.

The tube may extend linearly along the axis of the dispensing module beyond the cosmetic, and the dispensing module may include a foil cover removably disposed on the opening of the tube to seal the cosmetic within an interior of the tube. The repeating radial pattern of the cross-section of the stem may have a capture angle not greater than twenty degrees. The solid carrier may include at least one of a wax, an oil, and a glycerin. The cosmetic may include at least one of a lipstick, an eye liner, and an eye shadow. A bottom surface of the stem may be color coded with an indicia of a color of the cosmetic in the dispensing module.

In an aspect, a cosmetic dispensing system disclosed herein includes a dispensing module including: a solid cosmetic; a tube on a first end of the dispensing module, the tube surrounding at least a portion of the solid cosmetic, the tube having an opening to dispense the solid cosmetic; a stem on a second end of the dispensing module, the stem having a radially repeating cross-sectional shape with one or more radially varying features; and a dispensing mechanism, the dispensing mechanism including a mechanical actuator configured to advance the solid cosmetic through the opening by creating a linear motion along an axis of the dispensing module in response to a rotational movement of the stem relative to the tube.

The cosmetic dispensing system may include a container, where: the container includes a receiver shaped and sized to removably and replaceable receive the stem of the dispensing module; the receiver engages the stem against linear displacement with a draw force of at least one newton along the axis of the dispensing module; the receiver has an interior cross section shaped to receive the stem with a capture angle not exceeding twenty degrees, and to rotationally fix the container to the stem when coupled thereto; and the tube of the dispensing module extends from the container when the stem of the dispensing module is placed for use within the receiver of the container. The container may have a gripping region with a radius of at least twenty five millimeters. The cosmetic dispensing system may include a shell for the container, the shell formed of a low durometer grip material overmolded on the container. The low durometer grip material may have a Shore OO hardness of 30-95.

In an aspect, a container for a cosmetic dispenser disclosed herein includes: a top with a receiver for a dispensing module, the receiver having a radially repeating geometry with an intrinsic capture angle not exceeding forty-five degrees; a bottom, the receiver defining a channel through the container to the bottom of the container such that a bottom surface of a stem of the dispensing module is exposed through the bottom of the container when the dispensing module is placed for use in the container; and a gripping region on an exterior surface of the container, the gripping region formed by a shell of a low durometer material with a thickness of at least one millimeter.

The container may have a radius along at least a portion of the gripping region not less than twenty five millimeters. The radially repeating geometry may have an intrinsic capture angle not exceeding twenty degrees. The shell may be formed of a high grip material overmolded on the container. The low durometer material of the shell may have a Shore OO hardness of 30-9 The shell may have a thickness of 1-3 millimeters. The shell may be formed of at least one of a silicon, a rubber, or a thermoplastic. The container may include a cap, the cap configured to removably and replaceably couple to the container, and the cap including a reservoir shaped and sized to receive a tube of the dispensing module. The cap and the container may include a plurality of magnets in complementary positions to removably secure the cap to the container when placed for use on the container. The cap may include a second shell providing a second gripping surface for the cap when removing the cap from the container.

In an aspect, a container disclosed herein includes an interior with a receiver, where: the receiver includes an opening exposing a top portion of the interior on a top of the container, the top portion having a first cross-sectional shape; the receiver defines a channel in a bottom of the container, the channel connecting the top portion of the interior to a bottom surface of the container, the channel having a second cross-sectional shape different than the first cross-sectional shape; the second cross-sectional shape of the channel includes a radially repeating geometry with an intrinsic capture angle not exceeding twenty degrees; the container includes a gripping region on an exterior surface thereof, the gripping region including an overmolded shell of a low durometer material with a thickness of at least one millimeter; and the container has a radius along at least a portion of the gripping region not less than twenty millimeters.

The container may include a removable and replaceable cap for the container. The container may include a cosmetic dispenser removably and replaceably positioned in the receiver of the container, the cosmetic dispenser including a stem coupled to the channel of the receiver when placed for use in the receiver with a draw force that resists linear displacement of the stem relative to the channel. The draw force may include at least one of a frictional fit between the stem and the receiver and a magnetic coupling between a first magnet on the container and a second magnet on the cosmetic dispenser. The draw force may be at least ten newtons. The draw force may not be greater than fifty newtons. The cosmetic dispenser may have a tube for a cosmetic, the tube shaped and sized to fit within the top portion of the interior of the container, and where the gripping region of the container has a first radius around an axis of the cosmetic dispenser at least twice as large as a second radius of the tube of the cosmetic dispenser around the axis of the cosmetic dispenser. The stem of the cosmetic dispenser may be visible through the bottom of the container when the cosmetic dispenser is placed for use in the container, and the bottom surface of the stem may be color coded with an indicia of a color of a cosmetic in the cosmetic dispenser. The cosmetic dispenser may be a dispensing module for a fluid cosmetic, the dispensing module including: a dispenser, where the dispenser includes a cosmetic in a reservoir, the cosmetic including a pigment in a fluid carrier, and a tube on a first end of the dispenser, the tube including an opening to the reservoir and a threaded end portion, where the stem is on a second end of the dispenser, the stem defining a cross-section having a repeating radial pattern with a capture angle less than twenty degrees with the second cross-sectional shape of the channel of the receiver; and an applicator, where the applicator includes a top for the dispenser, the top including a threaded top portion complementary to the threaded end portion of the tube, where the top, when coupled to the dispenser by threading the threaded top portion to the threaded end portion, encloses the cosmetic within the reservoir of the dispenser, a wand extending from the top into the reservoir when the top is placed for use on the dispenser, and an applicator tip on the wand. The cosmetic dispenser may be a dispensing module for a solid cosmetic, the dispensing module including: a cosmetic; a tube on a first end of the dispensing module, the tube surrounding at least a portion of the cosmetic, and the tube having an opening to dispense the cosmetic; and a dispensing mechanism, the dispensing mechanism including a mechanical actuator configured to advance the cosmetic through the opening in the tube by creating a linear motion along an axis of the dispensing module in response to a rotational movement of the stem relative to the tube, where the stem is on a second end of the dispensing module, the stem having a radially repeating cross-sectional shape with a capture angle not greater than twenty degrees with the second cross-sectional shape of the channel of the receiver.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a cosmetic dispenser for a solid cosmetic.

FIG. 2 shows a cosmetic dispenser for a solid cosmetic.

FIG. 3 shows a cross-section of a cosmetic dispenser for a solid cosmetic.

FIG. 4 shows an exploded view of a cosmetic dispenser for a solid cosmetic.

FIG. 5 shows a cosmetic dispenser for a solid cosmetic.

FIG. 6 shows a geometry of a cross section for a receiver or a stem.

FIG. 7 shows a perspective view of a cosmetic dispenser for a solid cosmetic.

FIG. 8 shows a cosmetic dispenser for a fluid cosmetic.

FIG. 9 shows a cosmetic dispenser for a fluid cosmetic.

FIG. 10 shows a cross-section of a cosmetic dispenser for a fluid cosmetic.

FIG. 11 shows an exploded view of a cosmetic dispenser for a fluid cosmetic.

FIG. 12 shows a perspective view of a cosmetic dispenser for a fluid cosmetic.

### DETAILED DESCRIPTION

All documents mentioned herein are incorporated by reference in their entirety. References to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

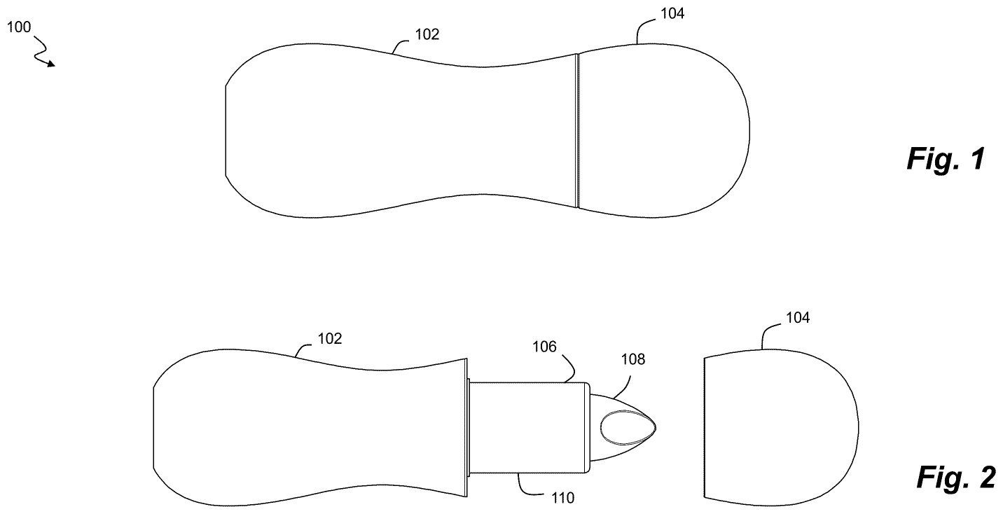

FIG. 1 shows a cosmetic dispenser for a solid cosmetic. In general, the cosmetic dispenser 100 may include a container 102 and a cap 104. In general, a solid cosmetic may be any cosmetic that retains a stable shape (at room temperature) in the absence of external forces. This may include any spreadable or deformable material having a hardness, spreadability, and viscosity suitable for application as a cosmetic. For a solid cosmetic may include any combination of mixtures, emulsions, gels, suspensions or the like using, e.g., matric of wax, oil(s), or the like to maintain structural stability. Typically (although not necessarily), a solid cosmetic may be any cosmetic that can be directly applied in solid form, e.g., as a pencil or similar application.

FIG. 2 shows a cosmetic dispenser for a solid cosmetic. The cosmetic dispenser 100 may be any of the cosmetic dispensers described herein. As depicted in FIG. 2, the cap 104 may be removable from the container 102 to expose a dispensing module 106 that is removably and replaceably disposed within the container 102. The dispensing module 106 may contain a cosmetic 108 including a solid cosmetic such as a lipstick, eye liner, eye shadow or the like that can be dispensed for use from the dispensing module 106. The cosmetic 108 may, for example, include a pigment in a solid carrier such as a wax, an oil, a glycerin, or some combination of these, as well as any other additives in or the like that might be used to control the consistency, fragrance, appearance, or other properties of the cosmetic 108, e.g., to improve the comfort, useability, durability, visual appeal, or healthiness of the cosmetic 108.

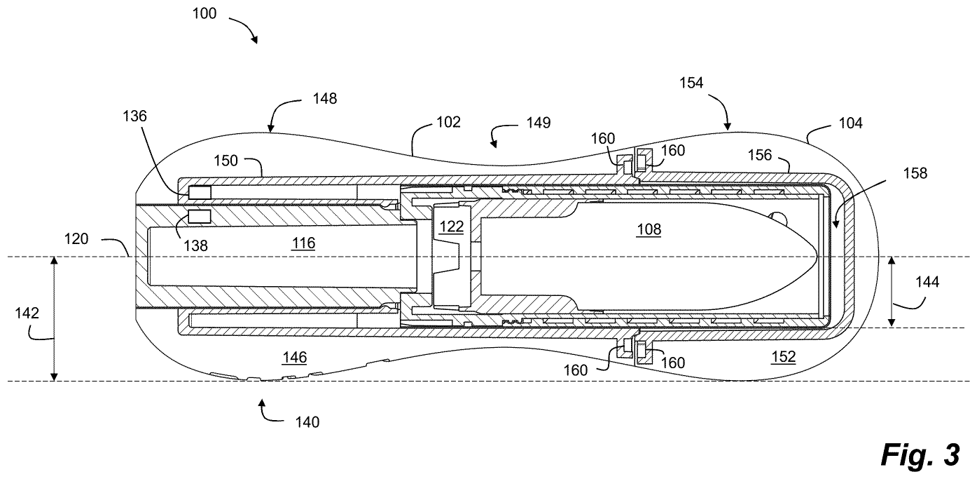

FIG. 3 shows a cross-section of a cosmetic dispenser for a solid cosmetic. The cosmetic dispenser 100 may be any of the cosmetic dispensers described herein. In general, the cosmetic dispenser 100, and the dispensing module 106 placed therein, may have an axis 120 extending through the container 102 and the cap 104. While the position of the axis 120 is not essential, the axis 120 is identified herein to provide a useful reference point for the alignment and movement of the various components of the cosmetic dispenser 100. For example, the cap 104 may be removed from the container 102 in a linear path along the axis 120, and the dispensing module 106 may be inserted into and removed from the container 102 in a linear path along the axis 120.

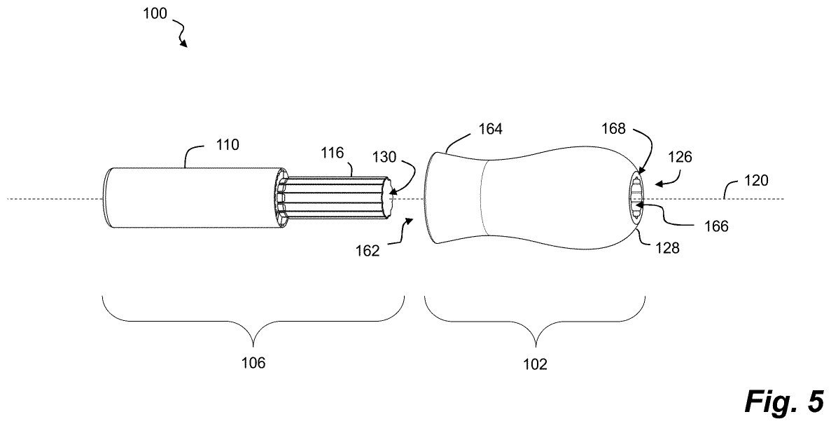

A dispensing mechanism 122 may be disposed within the dispensing module 106, and may include any mechanical actuator or combination of mechanical actuators configured to advance the cosmetic 108 from the tube 110 through the opening 114 of the dispensing module 106. In one aspect, the dispensing mechanism 122 may operate to create a linear motion along the axis 120 of the dispensing module in response to a rotational movement 124 of the stem 116 relative to the tube 110 (as illustrated in FIG. 5). Thus, when the stem 116 is rotated while the tube 110 is rotationally secured (e.g., where a user holds the exterior of the tube 110 where it extends from the container 102 while twisting the container 102), the cosmetic 108 may be advanced through the opening for use or retracted into the tube 110 for storage. A number of linear actuators are known in the art (of mechanics generally, and cosmetics packaging in particular) for converting rotational movement into linear movement. For example, the dispensing mechanism 122 may include a threaded screw on a central shaft, where either the shaft or the screw are linearly fixed so that rotation of the screw relative to the shaft generates linear forces to linearly displace the complementary component. In another aspect, the dispensing mechanism 122 may include a protrusion on a shaft, which travels within a helical groove of a surrounding sleeve or the like to generate linear motion as the protrusion travels within the groove. Because such actuators are well known, the dispensing mechanism 122 is not described in intricate detail here. However, generally, any threaded mechanism or other mechanical system or combination of systems that can be used to convert rotation of the stem 116 into linear advancement and retraction of the cosmetic 108 may be used as a dispensing mechanism 122 for the purposes described herein.

In one aspect, the container 102 may be overmolded with a shell 146 of a low durometer material or the like shaped and sized to increase the grippable radius of the cosmetic dispenser 100 and/or provide a soft surface for easier gripping and manipulation. In one aspect, the container 102 may have a gripping region 140 (e.g., provided by the shell 146) with a first radius 142 of at least twenty five millimeters. In another aspect, the first radius 142 may be at least twenty millimeters, or between twenty to twenty five millimeters, or between twenty five and thirty millimeters, or some other dimensions suitable for gripping by dexterity-challenged users. In another aspect, the gripping region 140 may have a first radius 142 at least twice as large as a second radius 144 of the tube 110 of the dispensing module 106.

The shell 146 may provide a gripping surface 148 for the container 102, e.g., in an area of the gripping region 140 and on other exterior surfaces of the container 102. The shell 146 may be formed of a relatively soft, high grip material overmolded on the container 102. For example, the shell 146 may be formed of a silicone, a rubber, a soft thermoplastic, or the like. The shell 146 may also or instead be formed of a low durometer material with a Shore OO hardness of 30-95. The shell 146 may also include other gripping features such as ridges, dots, protrusions, fins, finger grips, and so forth that make the container 102 easier to grip and manipulate.

The shell 146 may have a thickness of 1-3 millimeters, or some other thickness or range of thicknesses suitable for covering a rigid interior component 150 of the container 102 (which forms, e.g., the receiver 126, retains magnets for coupling to other components, and/or provides other structure to the container 102 to support mechanical functions thereof) and imparting a desired external shape to provide an ergonomic, comfortable, easy-to-grip surface for manipulation by a user. In general, the rigid interior component 150 may be fashioned of a hard plastic suitable for supporting various structures and functions of the cosmetic dispenser 100, while the shell 146 can provide an ergonomic exterior surface for manipulation by a user. For example, the shell 146 may be fabricated from a silicone, rubber, or other low durometer plastic or other synthetic material, while the rigid interior component 150 may be formed of any suitable hard plastic, such as Acrylonitrile Butadiene Styrene (ABS), Polycarbonate (PC), Polyamide (Nylon), Polyoxymethylene (POM), or Polyethylene Terephthalate (PET), or any other suitably rigid, durable material(s).

The cap 104 may also or instead be overmolded with a shell 152 providing a gripping surface 154 for the cap 104. Where the same material is used to overmold the cap 104 and the container 102, this may advantageously provide an aesthetically and functionally consistent surface for the entire cosmetic dispenser 100. The gripping surface 154 of the cap 104 may include any of the features, dimensions, materials, and the like described above for the shell 146 of the container 102. The cap 104 may be configured to removably and replaceably couple to the container 102. In one aspect, the shell 152 of the cap 104 may be overmolded to a rigid interior component 156 that includes a reservoir 158 shaped and sized to receive the tube 110 of the dispensing module 106. The rigid interior component 156 may be fabricated from any of the hard plastics described herein, or any other suitably rigid material to support the structure and functions of a cap 104 for a cosmetic dispenser. In one aspect, the rigid interior component 156 may form a loose interference fit with the tube 110 so that the cap 104 can be removed from the container 102 without disengaging the stem 116 from the receiver 126. In another aspect, magnets may be used to removably and replaceably couple the cap 104 to the container 102. For example, the cap 104 and the container 102 may include a plurality of magnets 160 in complementary positions to removably secure the cap 104 to the container 102 when placed for use. In one aspect, the magnets 160 may include individual magnets at a number of locations about the radius of the cap 104 and the container 102. In another aspect, ring magnets may be used that traverse the entire radius of the container 102 and/or cap 104, which advantageously permits omnidirectional radial orientation of the cap 104 and the container 102 when magnetically coupled together, and permits the cap 104 to be rotated about the axis 120 without magnetically decoupling the cap 104 and container 102. More generally, the plurality of magnets 160 in complementary positions may permit the cap 104 to be assembled to the container 102 even in the absence of a mechanically coupling infrastructure provided by, e.g., a friction fit between the tube 110 and the stem 116 of the dispensing module 106.

As a further advantage, the cap 104 and the container 102 may collectively form a volume with a narrower central radius (along the axis 120) at a midpoint 149 between the gripping surface 148 of the container 102 and the gripping surface 154 of the cap 104 in order to reduce the gripping force required by a user to linearly separate the cap 104 from the container 102. In this context, the midpoint 149 represents a general location along the axis 120 between the gripping surface 148 of the container 102 and the gripping surface 154 of the cap 104, rather than a precise mathematic or geometric midpoint therebetween.

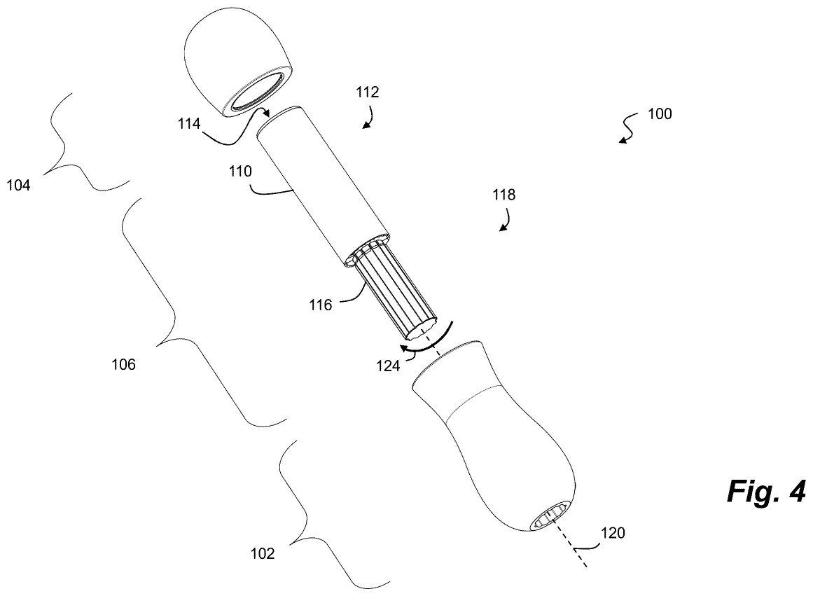

FIG. 4 shows an exploded view of a cosmetic dispenser for a solid cosmetic. The cosmetic dispenser 100 may be any of the cosmetic dispensers described herein. As illustrated in FIG. 3, the cap 104 may be removed from the container 102, and the dispensing module 106 may be removable from (and replaceable to) the container 102.

The dispensing module 106 may include a tube 110 on a first end thereof, the tube 110 containing a cosmetic 108 (not shown in FIG. 3), such as a solid cosmetic including a pigment in a solid carrier. The tube 110 may surround at least a portion of the cosmetic 108, e.g., some or all of a solid form of the cosmetic 108 may be disposed within and enclosed by an interior volume of the tube 110. The cosmetic 108 may be sealed in the tube 110 during manufacture and/or prior to sale by a foil, plastic sheet, screw top, or other scaling component that is secured over an opening 114 in the tube 110. This facilitates sale and delivery of the dispensing module 106 as a stand-alone, modular cosmetic component that can be unsealed by an end user (e.g., by removing the foil or other part) and placed into the container 102 for use as a cosmetic. The cosmetic 108 may then be dispensed as needed, and then covered with the cap 104 to protect the cosmetic 108 while it is not in use. The opening 114 may generally provide a physical path for dispensing the cosmetic 108 from the dispensing module 106.

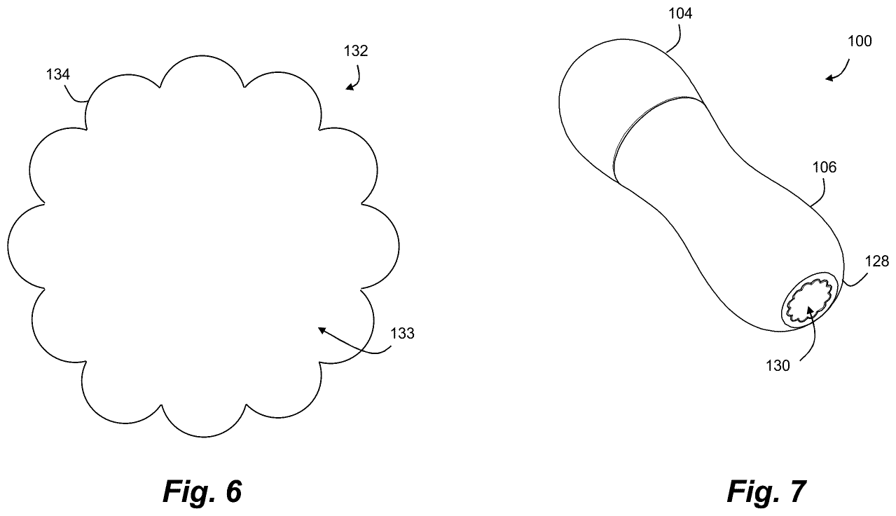

A stem 116 may extend from a second end 118 of the dispensing module 106. In this context, it will be understood that the first end 112 and the second end 118 of the dispensing module 106 refer to generally opposing end regions thereof, rather than specific locations or features on the dispensing module 106. In general, the stem 116 may have a cross-section with a non-circular shape, which facilitates rotational engagement and locking of the stem 116 relative to the container 102, e.g., so that a rotational force can be applied to the stem 116 from the container 102 in order to dispense the cosmetic 108. For example, the receiver 126 of the container 102 may constrain the stem 116 against relative rotational displacement with one or more radially varying features, such as the features 134 described with reference to FIG. 6.

FIG. 5 shows a cosmetic dispenser for a solid cosmetic. The cosmetic dispenser 100 may be any of the cosmetic dispensers described herein, and may include a dispensing module 106 and a container 102. In general, the dispensing module 106 may be removable from and replaceable to the container 102, e.g., so that a user can refill the cosmetic 108 or change a color or type of the cosmetic 108 as desired. To facilitate this change, the container 102 may include a receiver 126 defined by an interior volume within the container 102 that is shaped and sized to receive the stem 116 of the dispensing module 106. In one aspect, the receiver 126 may define a channel completely through the container 102 along the axis 120. More specifically, the receiver 126 may define a channel through the container 102 to a bottom 128 of the container 102 such that a bottom surface 130 of the stem 116 is visible through the bottom 128 of the container 102 when the dispensing module 106 is placed for use in the container 102.

The receiver 126 may engage the stem 116 against linear displacement along the axis 120 of the container 102, e.g., with a draw force sufficient to retain the dispensing module 106 in the receiver 126 during ordinary use, while permitting linear assembly and disassembly of the container 102 and the dispensing module 106, e.g., to replace the dispensing module 106. For example, the receiver 126 may engage the stem 116 against linear displacement with a draw force of at least one Newton along the axis 120 of the dispensing module 106 (and the container 102). This threshold provides a relatively gentle fit, while permitting the container 102 to be inverted without the stem 116 disengaging due to the forces of gravity. In another aspect, the draw force may be at least ten Newtons. In another aspect, the draft force does not exceed fifty Newtons. More generally, any draw force or range of draw forces may be used that retains the stem 116 in the receiver 126 during ordinary handling and use, while permitting assembly and disassembly without a need for tools or excessive manual strength.

In one aspect, the draw force may include a frictional fit between the stem 116 and the receiver 126, or any other interference fit, press fit or the like that resists linear displacement in a manner that retains the stem 116 in the receiver 126 during use, while permitting removal of the stem 116 from the receiver 126 and replacement of the stem 116 to the receiver 126 without undue force or specialized tools, e.g., with simple manipulation by a user's hands. While a friction fit can provide a useful temporary assembly for some materials, the engagement may also or instead be supported through the use of complementary magnets (or a magnet and a ferromagnetic material) on the receiver 126 and the stem 116, or some other snap fitting or the like that secures the relative position of the stem 116 and the receiver 126 when placed for use. Thus in one aspect, the draw force that resists linear displacement of the stem 116 may include a magnetic coupling between a first magnet 136 on the container 102 and a second magnet 138 on the dispensing module 106, or any other suitable combination or arrangement of magnets or the like.

In one aspect, the tube 110 of the dispensing module 106 may extend from the container 102 so that it is exposed to a user when the stem 116 of the dispensing module 106 is placed for use within the receiver 126 of the container 102. In this configuration, a user can rotate the stem 116 relative to the tube 110 by gripping an exposed portion of the tube 110 and rotating container 102 relative to the tube 110. This advantageously permits the use of the dispensing module 106 with a container 102 that extends the geometry of the cosmetic dispenser 100, e.g., to provide gripping regions with an expanded radius for easy manipulation, as well as a soft overmolding or the like to facilitate handling of the cosmetic dispenser 100 during use.

In one aspect, the interior of the container 102 may have an interior with at least two different cross-sectional shapes. While a view of the interior is occluded in FIG. 5, the interior may generally include an opening 162 that exposes a top portion 164 of the interior for insertion of the dispensing module 106 therein. As described herein, the receiver 126 may also include a channel 166 that connects this top portion 164 of the interior to a bottom surface 168 of the container 102 so that the bottom surface 130 of the stem 116 is visible from the exterior of the container 102 when the dispensing module 106 is placed for use in the container 102. In general, the interior may transition from a first cross-sectional shape, e.g., to receive the tube of the dispensing module 106, to a second cross-sectional shape to engage the stem 116. This second shape may include any of the radially varying cross-sectional geometries described herein for linearly accepting and rotationally engaging the stem 116 to permit operation of a cosmetic container. Although a solid cosmetic dispenser is illustrated in FIG. 5, it will be understood that a fluid cosmetic dispenser may use a similar interior configuration to receive and functionally engage the cosmetic dispenser for use.

FIG. 6 illustrates a geometry of a cross section for a receiver or a stem. In general, a receiver 126 of a container 102 for a cosmetic dispenser 100, such as any of the cosmetic dispensers described herein, may have a cross section 132 that is geometrically shaped to rotationally fix the container 102 to the stem 116 when coupled thereto. For example, the cross section 132 of the receiver 126 may include an interior 133 with features 134, such as twelve similarly shaped and regularly spaced protrusions or indents that can interchangeably receive a complementary geometry of the stem 116 in twelve different orientations. In this case, the exterior of the cross section 132 is the material of the container 102, and the interior 133 of the cross section 132 defines a void space to receive the stem 116. Conversely, the cross section 132 may be for the stem 116, in which case the interior 133 of the cross section 132 is the stem 116 itself, and the cross section 132 may enclose a exterior perimeter of the stem 116. In this case, the stem 116 may include twelve similarly shaped and regularly spaced features 134 such as teeth, vertices, or arcs that can interchangeably insert into a complementary geometry of the receiver 126 in twelve different orientations.

This arrangement advantageously provides a geometric interface that mechanically resists relative rotation between the parts, while providing a relatively small capture angle during assembly. In this context, the capture angle refers to the greatest possible rotational misalignment between two parts. For example, a rectangle (or an oval) will have two possible orientations to fit into a complementary rectangle (e.g., a rectangle with similar or identical dimensions, subject to minor variations to permit one to fit within the other, possible with a friction fit or the like to retain relative positions). In this case, the rectangle will rotate one-hundred eighty degrees between alignments, with a maximum possible misalignment of ninety degrees. Thus, two complementary rectangles have a capture angle of ninety degrees. Similarly, two equilateral triangles will have a capture angle of sixty degrees, and two squares will have a capture angle of forty-five degrees.

In another example, the cross section 132 of FIG. 6 provides twelve possible, symmetrically spaced alignments for a complementary geometry, which yields an angle of thirty degrees between recurring radial features, or a capture angle of fifteen degrees. Generally, a repeating radial geometry such as that illustrated in FIG. 6 may be used to design parts with a predetermined capture angle. More generally, a variety of cross sections, such as various complementary geometries (e.g., of the same shape, except as modified to permit one to fit within the other in a friction fit engagement or the like) and non-complementary (e.g., having different shapes with a number or contact features that permit multiple rotationally locked alignments) geometries, may be used to support rotational interlocking with a relatively small capture angle. In one aspect, e.g., as illustrated in FIG. 6, the cross section of the stem 116 may have a repeating radial pattern with a capture angle of less than twenty degrees, which usefully supports easy assembly with a small amount of rotational experimentation to engage the stem 116 to the receiver 126. Similarly, the receiver 126 may include a geometric void providing a capture angle of less than twenty degrees for the cross-section of the stem.

It will be noted that, in principle, various capture angles may be achieved with relatively arbitrary geometries for at least one of the interior and exterior radial components. For example, a receiver 126 may be configured with a radially repeating group of eighteen equally spaced indents with sixty degree vertices. This geometry could accommodate an equilateral triangle of suitable dimensions with a capture angle of ten degrees. Thus, in the context of this disclosure, a capture angle is either defined as a capture angle for a pair of specific parts, e.g., the stem 116 and the receiver 126 together as a pair, or, where a single part is recited, for that part and a complementary geometry. Thus, for example, where a capture angle is defined for just one part, e.g., a stem in isolation, then the capture angle is determined as the capture angle for that cross section 132 when inserted into (or received by) the same geometry (except to the extent that it must be slightly resized to facilitate such a physical assembly). So, for a stem 116 with a cross section 132 that is a square, and without a defined receiver, the capture angle will be forty-five degrees. This capture angle—for a single cross sectional shape rather than a pair of mated cross sections for a stem and receiver—is also referred to herein as an intrinsic capture angle.

In general, a geometry for the stem 116, the receiver 126, or the stem 116 and receiver 126 may usefully have a capture angle of not greater than forty-five degrees, note greater than twenty degrees, not greater than fifteen degrees, or any larger or smaller capture angle depending on the desired user experience when mating a stem 116 to a receiver 126. In general, a capture angle of between ten and twenty degrees appears to facilitate quick and easy assembly by a range of users.

In one aspect, the receiver 126 may rotationally fix the container 102 to the stem 116 to resist relative rotational displacement about the axis 120 against a torque of at least 2 Newton-centimeters. While mechanically interlocked pieces will resist rotational displacement until a mechanical yield or fracture point of the underlying materials, suitable rotational engagement such as this may also be achieved for many use cases with a circular geometry and a suitably strong friction fit. In one aspect, the stem 116 and/or the receiver 126 may be tapered to facilitate linear assembly and disassembly.

In another aspect, there is disclosed herein a stand-alone dispensing module for use with a cosmetic dispenser. The dispensing module may include a cosmetic including a pigment in a solid carrier; a tube on a first end of the dispensing module, the tube surrounding at least a portion of the cosmetic; an opening in the first end of the dispensing module; a stem extending along an axis of the dispensing module from a second end of the dispensing module, the stem defining a cross-section with a repeating radial pattern having a capture angle less than twenty degrees to a receiver with a complementary geometric shape; and an actuator to linearly dispense the cosmetic through the opening in response to a rotation of the stem relative to the tube. The tube may extend linearly along an axis of the dispensing module beyond the cosmetic, and may include a foil cover, plastic film, or other cap or cover removably disposed on the opening of the tube to seal the cosmetic within an interior of the tube, e.g., for sale and shipping of the dispensing module.

In another aspect, there is disclosed herein a stand-alone container for use with a dispensing module, the container including an interior with a receiver for a dispensing module, a gripping surface, wherein: the receiver has a radially repeating geometry that constrains a stem of the dispensing module with a complementary geometry against relative rotational displacement, and that receives the complementary geometry with a capture angle not exceeding twenty degrees, the receiver defines an opening in a bottom of the container, further wherein a bottom surface of the stem of the dispensing module is exposed through the opening when the dispensing module is placed for use in the container, and the container includes a gripping region on an exterior surface thereof, the gripping region including an overmolded shell of a low durometer material with a thickness of at least one millimeter, and the container having a radius along at least a portion of the gripping region not less than twenty or not less than twenty five millimeters. The container may also include a removable and replaceable cap for the container, which may be used to cover a cosmetic within the container, e.g., in a dispensing module placed in the container, when not in use.

In another aspect, a cosmetic dispenser described herein includes a dispensing module and a container. The dispensing module may include a cosmetic; a tube on a first end of the dispensing module, the tube surrounding at least a portion of the cosmetic, the tube having an opening to dispense the cosmetic; a stem on a second end of the dispensing module, the stem having a radially repeating cross-sectional shape, and a dispensing mechanism, the dispensing mechanism including a mechanical actuator configured to advance the cosmetic through the opening by creating a linear motion along an axis of the dispensing module in response to a rotational movement of the stem relative to the tube. The container may include a receiver shaped and sized to removably and replaceable receive the stem of the dispensing module, wherein the receiver engages the stem against linear displacement with a draw force of at least one Newton along an axis of the dispensing module, and further wherein the receiver has an interior cross section shaped to receive the stem with a capture angle not exceeding twenty degrees, and to rotationally fix the container to the stem when coupled thereto.

FIG. 7 shows a perspective view of a cosmetic dispenser for a solid cosmetic. The cosmetic dispenser 100, which may be any of the cosmetic dispensers described herein, may include a cap 104 and a container 102 enclosing a dispensing module 106. As noted above, a receiver 126 of the container 102 may define a channel through to a bottom 128 of the container 102, such that a bottom surface 130 of the stem 116 is visible through the bottom 128 of the container 102 when the dispensing module 106 is placed for use in the container 102. In this configuration, the bottom surface 130 of the stem 116 may be visually encoded with information about the contents of the dispensing module 106 such as a name of the cosmetic 108, a textual description of the color of the cosmetic 108, or a sample showing properties such as a color, a finish, and so forth of the cosmetic 108. For example, the bottom surface 130 may be color coded with an indicia of a color of the cosmetic 108 in the dispensing module 106, such as a replica of the cosmetic 108 as it would look when applied.

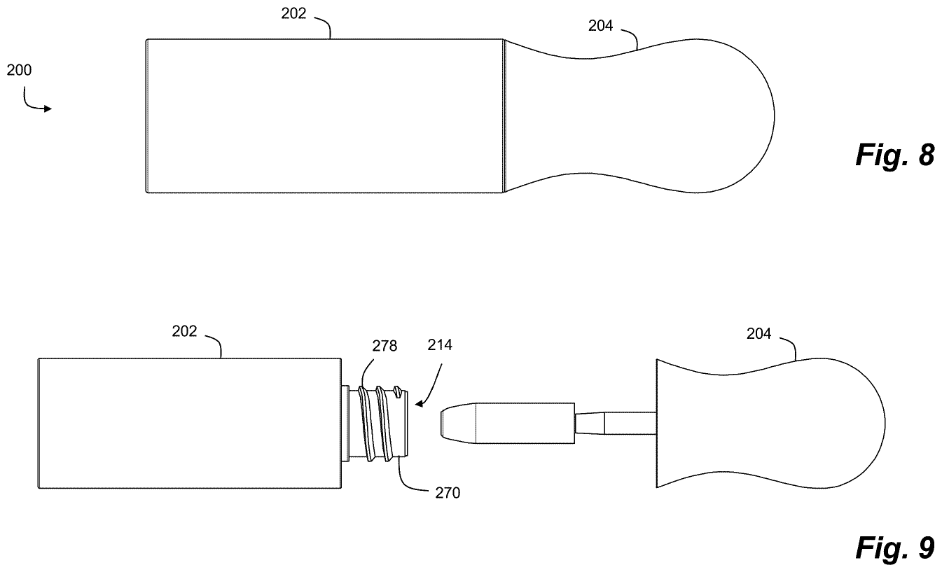

FIG. 8 shows a cosmetic dispenser for a fluid cosmetic. The cosmetic dispenser 200, which may be any of the cosmetic dispensers described herein except as specifically noted, may generally include a container 202 and a cap 204. In general, a fluid cosmetic may be any liquid or otherwise flowable cosmetic. This may include Newtonian fluids, non-Newtonian fluids, high-viscosity fluids, low-viscosity fluids, and so forth. Typically (although not necessarily), a fluid cosmetic may be any flowable cosmetic that can be applied, e.g., with a brush, swab, and the like, and/or that requires fluidic sealing to prevent leakage during storage. It will be appreciated that certain solid or semi-solid cosmetics may also be applied with a brush, and the nature of a dispenser for such cosmetics will depend on whether the mechanical properties of the material are more suitable for use in the self-supporting structure of a pencil dispenser or the like (e.g., such as the lipstick or other solid cosmetics described above) or with a brush or other applicator as depicted, e.g., in FIGS. 8 and 9.

FIG. 9 shows a cosmetic dispenser for a fluid cosmetic. The cosmetic dispenser 200 may be any of the cosmetic dispensers described herein, except where specifically noted otherwise. As depicted in FIG. 9, the cap 204 may be removable from the container 202 to expose a dispensing module 206 that is removably and replaceably disposed within the container 202. The dispensing module 206 may contain a cosmetic 208 including a fluid cosmetic that can be dispensed for use from the dispensing module 106. For example, the cosmetic 208 may include a nail polish, a lip gloss, a mascara, an eye liner, or any other cosmetic formed of a pigment in a fluid carrier (or, where no pigment is used, simply the fluid carrier and additives). The cosmetic 208 may also include ingredients such as an emulsifier, a preservative, a thickener, and/or an emollient, as well as any other additives or the like that might be used to control the consistency, fragrance, appearance, or other properties of the cosmetic 108, e.g., to make the cosmetic 108 more comfortable, easy, durable, healthy, or appealing for a user. It will be noted that where the cosmetic dispenser 200 is a fluid cosmetic dispenser, the dispensing module 206 may include two separate parts, a dispenser 270 that is removably and replaceably coupled to the container 202, and an applicator 280 that is removably and replaceably coupled to the cap 204 of the cosmetic dispenser 200. These parts (the dispenser 270, the applicator 280, and the dispensing module 206 formed by the assembled dispenser 270 and applicator 280) are generally referred to interchangeably herein, except where it is necessary to specify one or both of the component parts, or where it is otherwise helpful to clarify an aspect of the cosmetic dispenser 200.

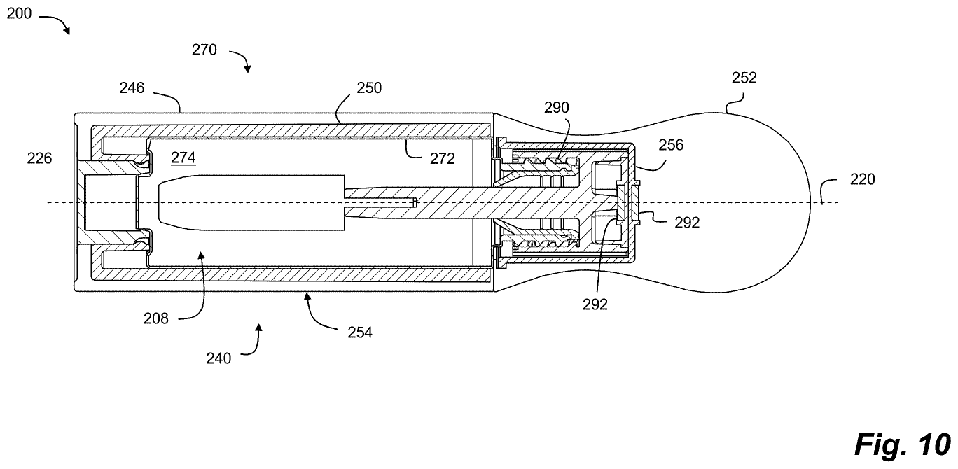

FIG. 10 shows a cross section of a cosmetic dispenser. The cosmetic dispenser 200 may be any of the cosmetic dispensers described herein. The dispenser 270 may include a tube 272 with a reservoir 274 containing the cosmetic 208. A first end 276 of the dispenser 270 of the dispensing module 206 may include an opening 214 to the reservoir 274 and a threaded end portion 278. A second end 282 of the dispenser 270 of the dispensing module 206 may have a stem 216 extending along an axis 220 of the cosmetic dispenser 200 (and the dispenser 270). In general, the first end 276 and the second end 282 refer generally to the relatively opposing end regions of the dispenser 270, rather than precise locations or extremities.

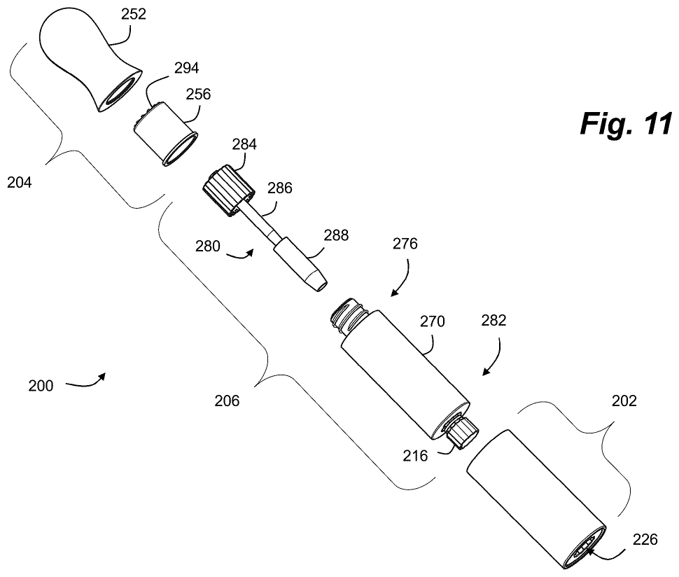

FIG. 11 shows an exploded view of a cosmetic dispenser for a fluid cosmetic. In general, the stem 216 of the dispensing module 206 may be any of the stems described herein, and may have a cross-sectional geometry suitable for engaging the container 202 of the cosmetic dispenser 200 in a rotationally fixed manner, while coupling to a receiver 226 of the container 202 with a draw force permitting removal of the dispenser 270 from the container 202 and replacement of the dispenser 270 to the container 202 via a linear motion along the axis 220 of the cosmetic dispenser 200. In this manner, the stem 216 may engage with the container 202 to provide a non-permanent functional coupling therebetween, all as more generally describe herein. For example, the stem 216 may have a cross-section with a non-circular shape. The cross-section of the stem 216 may also or instead have a repeating radial pattern with a capture angle of less than twenty degrees, or less than twenty five degrees. The receiver 226 may also or instead have a geometric void providing a capture angle of less than twenty degrees, or less than twenty five degrees, for the cross-section of the stem 216. The receiver 226 may geometrically constrain the stem 216 against relative rotational displacement with one or more radially varying features. The receiver 226 may also or instead rotationally fix the container 202 to the stem 216 to resist relative rotational displacement against a torque of at least 2 Newton-centimeters. It will be understood that while this rotational engagement supports dispensing of a solid cosmetic in solid cosmetic embodiments of a cosmetic dispenser, e.g., for lipstick, the rotational engagement may alternatively support capping and uncapping of a threaded top for the dispenser 270 in fluid cosmetic embodiments of a cosmetic dispenser 200, e.g., for a mascara, lip gloss, eye liner, or the like.

In order to secure the dispenser 270 once placed for use in the container 202, the stem 216 may removably and replaceably engage the receiver 226 with a friction fit or the like providing a draw force that resists linear displacement of the stem 216 relative to the receiver 226. In one aspect, the draw force may be between one Newton and ten Newtons. In another aspect the draw force may be a force not exceeding fifty Newtons. The draw force may also or instead include a magnetic coupling between a first magnet on the container 202 and a second magnet on the dispensing module 206, or more specifically, a second magnet on the dispenser 270 of the dispensing module 206. While the positions of such magnets are not illustrated, it will be understood that the cosmetic dispenser 200 may use similar arrangements of magnets as those described above for the cosmetic dispenser 100 for a solid cosmetic.

The applicator 280 of the dispensing module 206 may include a top 284, a wand 286, and an applicator tip 288. The top 284 for the applicator 280 of the dispensing module 206 may include a threaded top portion 290 complementary to the threaded end portion 278 of the dispenser 270 of the dispensing module 206. The top 284, when coupled to the dispenser 270 of the dispensing module 206 by threading the threaded top portion 290 of the top 284 to the threaded end portion 278 of the dispenser 270, may enclose the cosmetic 208 within the reservoir 274 of the dispensing module 206, and fluidically seal the cosmetic 208 in the reservoir 274. The wand 286 may extend from the top 284 into the reservoir 274 when the top 284 is placed for use on the dispenser 270 of the dispensing module 206. The applicator tip 288 may be disposed on the wand 286, and provides a functional surface for dispensing and applying the cosmetic 208 from the reservoir 274. The applicator tip 288 may, for example, include a brush, a sponge, a swab, and so forth, and may be configured for use in applying nail polish, eye liner, lip gloss, mascara, and so forth. As is known in the art, the opening 214 may also include any combination of brushes, wipers, drainage channels and the like to facilitate drip free dispensing of the cosmetic 208 on the applicator tip 288 during use.

The container 202 may be any of the containers described herein. In general, the container 202 may provide a removable and replaceable exterior for the dispensing module 206 to facilitate improved useability, e.g., by dexterity-challenged users. In general, the dispensing module 206, or more specifically, the dispenser 270 of the dispensing module 206, may be removable from and replaceable to the container 202 using any of the techniques described herein to engage the stem 216 of the dispenser 270 with the receiver 226 of the container 202.

As described in various embodiments herein, the receiver 226 may be shaped and sized to receive the stem 216 of the dispensing module 206, or more specifically, of the dispenser 270 of the dispensing module. The receiver 226 may engage the stem 216, when placed for use therein, against linear displacement with a draw force of at least one Newton, or between one Newton and ten Newtons, along an axis of the dispensing module 206. The receiver 226 may be shaped and sized to rotationally fix the container to the stem when coupled thereto. To facilitate capping and uncapping of the dispenser 270, the threaded end portion 278 of the (dispenser 270 of) the dispensing module 206 may extend from the container 202 (e.g., as illustrated in FIG. 9) when the stem 216 of the dispensing module 206 is placed for use into the receiver 226 of the container 202.

The receiver 226 may be formed in a rigid interior component 250 of the container 202, which may be fabricated from any of the hard plastics described herein, or any other suitably rigid material to support the structure and function of the container 202. The rigid interior component 250 of the container 202 may, for example, provide a channel with a suitable geometry to receive the stem 216 of the dispenser 270. A shell 246 of a low durometer material may be fabricated around an exterior of the rigid interior component 250 to facilitate manipulation of the cosmetic dispenser 200, and to provide a gripping surface 254 for the container 202. The cap 204 of the cosmetic dispenser 200 may also include a rigid interior component 256 to support the structure and function of the cap 204, and the rigid interior component 256 of the cap 204 may also be formed of a hard plastic or other suitably rigid material. The cap 204 may also be overmolded with a shell 252 such as any of the exterior shells described herein.

As further described in various embodiments, the container 202 may include various structural features to improve useability and ease of handling for the cosmetic dispenser 200. This may include any of the features of a cosmetic dispenser 100 for solid cosmetics as described above, or any other suitable features or the adaptations thereof for the cosmetic dispenser 200 for fluid cosmetics. In one aspect, the container 202 may have a gripping region 240 (e.g., formed into the shell 246) with a radius of at least twenty millimeters, or at least twenty five millimeters. In another aspect, the container 202 may have a gripping region 240 with a radius at least twice the radius of the tube 272 of the dispenser 270.

In general, the shells 246, 252 for the cosmetic dispenser 200 for a fluid cosmetic may be the same as the shells 146, 152 for the cosmetic dispenser 100 for solid cosmetics, except as specifically noted otherwise. For example, the shell 246 for the container 202 and/or the shell 252 for the cap 204 may be formed of a relatively soft high grip material overmolded onto the container 202 or the cap 204, respectively. The shell 246, 252 may be formed of a low durometer material with a Shore OO hardness of 30-95. The shells 246, 252 may have a thickness of 1-3 millimeters. The shells 246, 252 may be formed of at least one of a silicon, a rubber, or a thermoplastic.

The cap 204 for a fluid cosmetic dispenser may differ in certain aspects from the cap 104 for a solid cosmetic dispenser. In general, this relates to differences in the manner in which the cosmetic dispensers 100, 200 are used, and the need for a fluidic seal for the reservoir 274 of the dispenser 270. To achieve this fluidic seal, the cap 204 may support threaded operation of the applicator 280, which threads onto the dispenser 270 to seal a fluid cosmetic in the reservoir 274 when not in use. In one aspect, the top 284 of the applicator 280 may slidably engage with the rigid interior component 256 of the cap 204, and may include one or more radial features to rotationally engage the rigid interior component 256, such as fins, protrusions, or the like. This facilitates opening of the dispenser 200 by applying a rotational force to unscrew the cap 204 (and the applicator 280 coupled thereto) from the dispenser 270, while the container 202, which is engaged to the stem 216 of the dispenser 270, can support a counter-rotational force on the threaded end portion 278.

To support threaded capping and uncapping, an exterior surface of the top 284 of the applicator 280 may define a cross-section with a repeating radial pattern having a capture angle of less than twenty degrees to the cap 204 (or more specifically, the rigid interior component 256 of the cap 204). More generally, the cap 204 may include a rigid interior component 256 shaped and sized to engage in a rotationally fixed manner with the top 284 of the applicator 280. The cap 204 may also include a gripping region of a relatively soft high grip material overmolded onto the rigid interior component 256 to providing a gripping surface for a user.

The top 284 may linearly engage with the rigid interior component 256 of the cap 204 with a friction fit or interference fit, and/or with one or more magnets 292 in complementary positions to secure the top 284 to the rigid interior component 256 for use. For example one of the magnets 292 may be positioned in a top surface of the top 284, while another one of the magnets 292 may be positioned in a complementary location on or near an interior surface of the rigid interior component 256, e.g., as illustrated in FIG. 10. In one aspect, the rigid interior component 256 of the cap 204 may engage the top 284 of the applicator 280 against linear displacement along the axis 220 of the dispensing module 206 with a draw force of at least one Newton, or at least five Newtons, or at least ten Newtons. More generally, the draw force may be selected (e.g., by suitable selection and arrangement of magnets, by suitably dimensioning a friction fit, and the like) to be sufficiently strong to retain the applicator 280 in the cap 204 during linear manipulation such as extracting the applicator tip 288 and applying the cosmetic 208, but sufficiently week that the cap 204 can be easily separated from the applicator 280 when the user wishes to change the dispensing module 206 in the cosmetic dispenser 200.

The rigid interior component 256 may include any number of exterior features 294 such as radial ridges on a top surface thereof that rotationally engage an overmolded material to improve rotational engagement therebetween, and to retain the rotational engagement of the shell 252 to the rigid interior component 256 over repeated rotational stresses (e.g., due to multiple uncapping and capping actions with the cosmetic dispenser 200 over time).

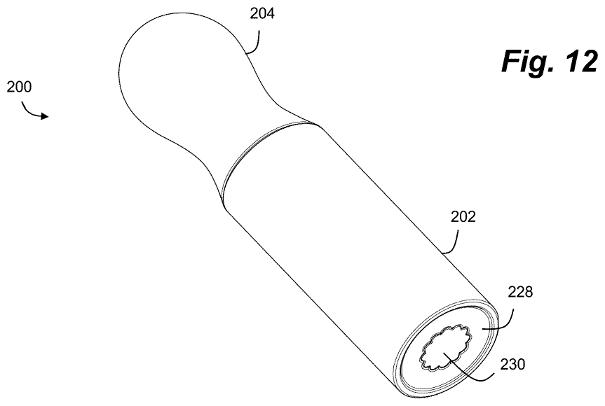

FIG. 12 shows a perspective view of a cosmetic dispenser for a fluid cosmetic. As described herein, when the receiver 226 defines a channel through the container 202 to a bottom 228 thereof and the dispensing module 206 is placed for use in the container 202, a bottom surface 230 of the stem 216 may be exposed and visible through the bottom 228 of the container 202, which advantageously provides a convenient, visible exterior surface for providing information about the nature of the cosmetic 208 in the dispenser 270. For example, the bottom surface 230 of the stem 216 may be color coded with an indicia of the color of the cosmetic in the dispensing module, or otherwise labeled with information about the type and color of the cosmetic 208.

According to the foregoing, there is also disclosed herein a stand-alone cosmetic dispenser including a dispenser and an applicator. The dispenser may include a cosmetic in a reservoir, the cosmetic including a pigment in a fluid carrier, a tube on a first end of the dispenser, the tube including an opening to the reservoir and a threaded end portion, and a stem extending along an axis of the dispenser from a second end of the dispenser, the stem, the stem defining a cross-section with a repeating radial pattern having an intrinsic capture angle less than twenty degrees. The applicator may include a top for the dispenser, the top including a threaded top portion complementary to the threaded end portion of the tube, wherein the top, when coupled to the dispenser by threading the threaded top portion to the threaded end portion, encloses the cosmetic within the reservoir of the dispenser, a wand extending from the top into the reservoir when the top is placed for use on the dispenser, and an applicator tip on the wand. An exterior surface of the top may define a second cross-section with a second repeating radial pattern having a second capture angle less than twenty degrees to a cap for the top.

In another aspect, there is disclosed herein a stand-alone container for a cosmetic dispenser including an interior with a receiver for a dispensing module, a gripping surface, wherein: the receiver has a radially repeating geometry having an intrinsic capture angle not exceeding twenty degrees, the receiver defines a channel through the container to a bottom of the container such that a bottom surface of the stem of the dispensing module is exposed through the bottom of the container when the dispensing module is placed for use in the container, and the container includes a gripping region on an exterior surface thereof, the gripping region including an overmolded shell of a low durometer material with a thickness of at least one millimeter, and the container having a radius along at least a portion of the gripping region not less than twenty five millimeters. The container may also include a removable and replaceable cap for the container.

In another aspect, there is disclosed herein a cosmetic dispenser including a dispenser, an applicator, and a container. The dispenser may include: a cosmetic; a tube on a first end of the dispenser, the tube including a reservoir for the cosmetic, an opening to the reservoir, and a threaded end portion; and a stem on a second end of the dispenser, the stem extending along an axis of the dispenser, and the stem having a radially repeating cross-sectional shape. The applicator may include: a top for the dispenser; a threaded top portion of the top with threads complementary to the threaded end portion of the tube, wherein the top, when coupled to the dispenser by threading the threaded top portion to the threaded end portion, encloses the cosmetic within the reservoir of the dispenser; and a wand with an applicator extending from the top into the reservoir when the top is placed for use on the dispenser. The container may include a receiver shaped and sized to removably and replaceable receive the stem of the dispenser, wherein the receiver engages the stem against linear displacement with a draw force of at least one Newton along an axis of the dispenser, and the receiver may have an interior cross section shaped to receive the stem with a capture angle not exceeding twenty degrees, and to rotationally fix the container to the stem when coupled thereto.

The method steps described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will also be appreciated that the methods and systems described above are set forth by way of example and not of limitation. Numerous variations, additions, omissions, and other modifications will be apparent to one of ordinary skill in the art. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A container for a cosmetic dispenser, the container comprising:
a top with a receiver for a dispensing module, the receiver having a radially repeating geometry with an intrinsic capture angle not exceeding forty-five degrees;
a bottom, the receiver defining a channel through the container to the bottom of the container such that a bottom surface of a stem of the dispensing module is exposed through the bottom of the container when the dispensing module is placed for use in the container; and
a gripping region on an exterior surface of the container, the gripping region formed by a shell of a low durometer material with a thickness of at least one millimeter.

2. The container of claim 1, wherein the container has a radius along at least a portion of the gripping region not less than twenty five millimeters.

3. The container of claim 1, wherein the radially repeating geometry has an intrinsic capture angle not exceeding twenty degrees.

4. The container of claim 1, wherein the shell is formed of a high grip material overmolded on the container.

5. The container of claim 1, wherein the low durometer material of the shell has a Shore OO hardness of 30-95.

6. The container of claim 1, wherein the shell has a thickness of 1-3 millimeters.

7. The container of claim 1, wherein the shell is formed of at least one of a silicon, a rubber, or a thermoplastic.

8. The container of claim 1, further comprising a cap, the cap configured to removably and replaceably couple to the container, and the cap including a reservoir shaped and sized to receive a tube of the dispensing module.

9. The container of claim 8, wherein the cap and the container include a plurality of magnets in complementary positions to removably secure the cap to the container when placed for use on the container.

10. The container of claim 8, wherein the cap includes a second shell providing a second gripping surface for the cap when removing the cap from the container.

11. A container, the container including an interior with a receiver, wherein:
the receiver includes an opening exposing a top portion of the interior on a top of the container, the top portion having a first cross-sectional shape,
the receiver defines a channel in a bottom of the container, the channel connecting the top portion of the interior to a bottom surface of the container, the channel having a second cross-sectional shape different than the first cross-sectional shape,
the second cross-sectional shape of the channel includes a radially repeating geometry with an intrinsic capture angle not exceeding twenty degrees,
the container includes a gripping region on an exterior surface thereof, the gripping region including an overmolded shell of a low durometer material with a thickness of at least one millimeter, and
the container has a radius along at least a portion of the gripping region not less than twenty millimeters.

12. The container of claim 11, further comprising a removable and replaceable cap for the container.

13. The container of claim 11, further comprising a cosmetic dispenser removably and replaceably positioned in the receiver of the container, the cosmetic dispenser including a stem coupled to the channel of the receiver when placed for use in the receiver with a draw force that resists linear displacement of the stem relative to the channel.

14. The container of claim 13, wherein the draw force includes at least one of a frictional fit between the stem and the receiver and a magnetic coupling between a first magnet on the container and a second magnet on the cosmetic dispenser.

15. The container of claim 13, wherein the draw force is at least ten Newtons.

16. The container of claim 13, wherein the draw force is not greater than fifty Newtons.

17. The container of claim 13, wherein the cosmetic dispenser has a tube for a cosmetic, the tube shaped and sized to fit within the top portion of the interior of the container, and wherein the gripping region of the container has a first radius around an axis of the cosmetic dispenser at least twice as large as a second radius of the tube of the cosmetic dispenser around the axis of the cosmetic dispenser.

18. The container of claim 13, wherein the stem of the cosmetic dispenser is visible through the bottom of the container when the cosmetic dispenser is placed for use in the container, and wherein the bottom surface of the stem is color coded with an indicia of a color of a cosmetic in the cosmetic dispenser.

19. The container of claim 13, wherein the cosmetic dispenser is a dispensing module for a fluid cosmetic, the dispensing module comprising:
a dispenser, wherein the dispenser includes:
a cosmetic in a reservoir, the cosmetic including a pigment in a fluid carrier, and
a tube on a first end of the dispenser, the tube including an opening to the reservoir and a threaded end portion, wherein
the stem is on a second end of the dispenser, the stem defining a cross-section having a repeating radial pattern with a capture angle less than twenty degrees with the second cross-sectional shape of the channel of the receiver; and
an applicator, wherein the applicator includes:
a top for the dispenser, the top including a threaded top portion complementary to the threaded end portion of the tube, wherein the top, when coupled to the dispenser by threading the threaded top portion to the threaded end portion, encloses the cosmetic within the reservoir of the dispenser,
a wand extending from the top into the reservoir when the top is placed for use on the dispenser, and
an applicator tip on the wand.

20. The container of claim 13, wherein the cosmetic dispenser is a dispensing module for a solid cosmetic, the dispensing module comprising:
a cosmetic,
a tube on a first end of the dispensing module, the tube surrounding at least a portion of the cosmetic, and the tube having an opening to dispense the cosmetic, and
a dispensing mechanism, the dispensing mechanism including a mechanical actuator configured to advance the cosmetic through the opening in the tube by creating a linear motion along an axis of the dispensing module in response to a rotational movement of the stem relative to the tube, wherein
the stem is on a second end of the dispensing module, the stem having a radially repeating cross-sectional shape with a capture angle not greater than twenty degrees with the second cross-sectional shape of the channel of the receiver.
